(19) United States
(12) Patent Application Publication
(10) Pub. No.: US 2010/0166844 A1
(43) Pub. Date: Jul. 1, 2010

(54) POLYMER PARTICLE DISPERSION, COMPOSITION CONTAINING THE SAME AND COSMETIC TREATMENT METHOD

(76) Inventors: Nathalle Mougin, Paris (FR); Gwenaëlle Jegou, Saint Michel Sur Orge (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/527,641

(22) PCT Filed: Feb. 12, 2008
(86) PCT No.: PCT/FR08/50213

§ 371 Date: Feb. 18, 2010

Related U.S. Application Data
(60) Provisional application No. 60/904,175, filed on Mar. 1, 2007.

(30) Foreign Application Priority Data
Feb. 19, 2007 (FR) 0753336

(51) Int. Cl. A61K 8/72; A61K 9/127; A61K 8/81; A61K 8/18; A61Q 5/04; A61Q 5/06; A61Q 5/08; A61Q 17/04; A61Q 5/02

(52) U.S. Cl. 424/450; 424/59; 424/62; 424/70.2; 424/70.11; 424/78.02; 424/78.31; 8/405; 510/119; 510/158

(57) ABSTRACT

The present patent application relates to novel dispersions of particles of polymer stabilized at the surface in a nonaqueous medium, in which dispersions the polymer comprises mainly hydrophilic cationic monomers.

Another subject-matter of the invention is a cosmetic or pharmaceutical composition comprising the said dispersion in a cosmetically or pharmaceutically acceptable medium.

The invention also relates to a method for the cosmetic treatment of keratinous substances employing the said composition.

The present invention relates to a novel dispersion of particles of highly specific polymers which are dispersed in a nonaqueous medium and to compositions, in particular cosmetic or pharmaceutical compositions, comprising the said dispersion.

Mention may be made, among the polymers employed in the cosmetics field and more particularly in the hair field, of cationic polymers, such as those based on dimethyldiallylammonium chloride, known in particular for protecting and/or beautifying the hair, by virtue of their high substantivity. However, no effect of shaping the hair is observed when polymers of this type are employed. Furthermore, their incompatibility with the majority of propellants does not allow them to be used in aerosol products, such as lacquers.

Hair compositions comprising aqueous dispersions of hydrophobic cationic polymers which contribute styling properties, for example when they are employed in shampoos, are known from EP 1 323 753. However, these compositions do not exhibit a very good cosmetic behaviour in a wet environment, in particular during applications of shampoos.

In order to obtain a good styling effect while retaining acceptable cosmetic properties in the compositions, provision has been made for polymers having styling properties which are carried in a cosmetic solvent. Thus, the documents WO 91/15185 and WO 98/18433 provide for cosmetic compositions comprising hydrophobic or water-insoluble polymers carried in solution in an organic solvent. However, the need to employ polymers which are soluble in an organic medium implies that the chemical structures available are of low variability.

Furthermore, it has been found that organic solutions of hydrophobic polymers generally exhibit a high viscosity, related to the polymer content of the solution, which complicates the subsequent formulation of these polymers and of their solutions.

Furthermore, the use is known, in cosmetics, of dispersions of polymer particles, generally of nanometric size, in organic media and in particular, through EP 749 747, of nonaqueous dispersions of particles of polymethyl acrylate or polymethyl methacrylate in a nonvolatile liquid paraffin or in isododecane, for example. However, these dispersions do not make it possible to obtain satisfactory cosmetic properties, in particular cosmetic properties which are satisfactory in terms of styling effect and of disentangling. Furthermore, the feel of the compositions comprising them may prove to be rather unsatisfactory, in particular with regard to the softness.

The Applicant Company has discovered, surprisingly, novel dispersions of polymer particles, stabilized by stabilizing agents, in nonaqueous media, which make it possible to contribute the desired cosmetic properties (feel, softness, disentangling) while improving the styling properties.

The presence of cationic monomers in the polymer in dispersion makes it possible in particular to contribute greater softness and to improve the disentangling; the deposition on the hair is more efficient, which is particularly advantageous in the case of damaged hair. Furthermore, the presence of cationic monomers makes it possible to improve the hold of the curl, that is to say the styling effect.

A subject-matter of the invention is thus a dispersion of particles of at least one ethylenic polymer, stabilized at the surface by a stabilizing agent, in a nonaqueous medium composed of at least one nonaqueous compound which is liquid at 25° C. and which has an overall solubility parameter, according to the Hansen solubility space, of less than or equal to 20 (MPa)^{1/2}, or a mixture of such compounds, characterized in that the said ethylenic polymer comprises from 50 to 100% by weight of hydrophilic cationic monomer having a log p of less than or equal to 2, or of a mixture of such monomers, with respect to the total weight of monomers.

Another subject-matter of the invention is a cosmetic or pharmaceutical composition comprising, in a cosmetically or pharmaceutically acceptable medium, at least one dispersion as defined above.

The dispersions according to the invention make it possible to obtain a satisfactory level of care and of disentangling which is superior to that of the prior art and which is lasting over time.

The invention makes it possible to prepare polymers which can be easily carried, given that the dispersions have relatively low viscosities, which facilitates the use thereof in cosmetic compositions.

Furthermore, these compositions make it possible to confer body and hold on the hair.

In addition, these dispersions or the compositions comprising them contribute advantageous properties, in particular in rinse-out mode. They make it possible to obtain, in addition to the styling effects and the hold, a soft and nonsticky feel, good softness and also ease of disentangling of the hair, in a dry and/or wet environment.

These polymers can also be used in “leave-in” products, such as hair care products (hair mask) or styling lacquer or styling gels/foams, in order to contribute, in addition to styling, cosmetic quality to the hair (feel, softness, smoothing, disentangling). They can also be introduced into compositions for pre- or post-treatment after hair dyeing, perming or straightening.

Another advantage of the invention lies in the fact that the polymer particles can be very small in size, in particular nanometric in size, which is not the case with, for example, other types of particles, such as microspheres, the diameter of which is generally greater than 1 micron. In point of fact, a large size of the order of a micron has the disadvantage of resulting in a degree of visibility of the particles to the eye, when they are in a composition and when they are applied to the skin, and also in poor stability of the composition, in particular over time. Thus, the dispersions according to the invention make it possible to obtain stable compositions which can in addition be transparent, translucent or opaque, according to preference, depending on the size of the polymer particles which are dispersed therein.

The dispersions according to the invention are thus composed of particles, generally spherical particles, of at least one ethylenic polymer, stabilized at the surface by a stabilizing agent, in a nonaqueous medium.

The dispersions according to the invention can in particular be provided in the form of polymer nanoparticles in stable dispersion in a nonaqueous medium. The nanoparticles preferably have a size of between 5 and 600 nm, in particular from 10 to 500 nm, better still from 15 to 450 nm, given that, beyond approximately 600 nm, the dispersions of particles become much less stable.

In particular, these particles remain in the state of individual particles, without forming agglomerates, when they are in dispersion in the said nonaqueous media.

“Ethylenic” polymer is understood to mean a polymer obtained by polymerization of at least 2 identical or different monomers comprising an ethylenic unsaturation.

The said ethylenic polymer can be chosen by a person skilled in the art as a function of its properties, according to the subsequent application desired for the composition. These polymers can in particular be crosslinked.

The polymers according to the invention can be homopolymers or copolymers which are linear, branched, grafted or even star-shaped. They can be random or alternating. Preferably, these are random linear copolymers.

The ethylenic polymer according to the invention comprises from 50 to 100% by weight of hydrophilic cationic monomer, or of a mixture of such monomers, with respect to the total weight of monomers. It preferably comprises from 51 to 99.5% by weight, better still from 52 to 99% by weight, indeed even from 55 to 98% by weight, in particular from 60 to 95% by weight, preferably from 65 to 85% by weight, of hydrophilic cationic monomer, alone or as a mixture, with respect to the total weight of starting monomers.

The hydrophilic cationic monomers are preferably monofunctional, that is to say that they preferably comprise only a single polymerizable functional group, in particular a vinyl functional group.

“Cationic monomer” is understood to mean any monomer having a charge when it is placed in an aqueous medium, at a pH of between 3 and 12.

“Hydrophilic monomer” is understood to mean, within the meaning of the present invention, a monomer having a value of the logarithm of the 1-octanol/water apparent partition coefficient, also known as log p, of less than or equal to 2, for example between −10 and 2, preferably of between −5 and 1.9 and in particular of between −2.5 and 1.5.

The log p values are known and are determined according to a standard test which determines the concentration of the monomer in 1-octanol and water.

The values can in particular be calculated using ACD (Advanced Chemistry Development) software Solaris V4.67; they can also be obtained from Exploring QSAR: Hydrophobic, Electronic and Steric Constants (ACS Professional Reference Book, 1995). There also exists an Internet site which provides estimated values (address: http://esc.syrres.com/interkow/kowdemo.htm).

Mention may be made, among the hydrophilic cationic monomers capable of being employed in the context of the invention, alone or as a mixture, of the monomers of following formulae (I) and (II), and their salts:

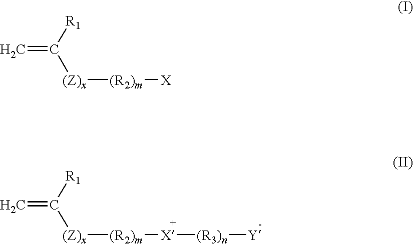

in which:
R₁ is a hydrogen atom or a linear or branched hydrocarbon radical of CₚH₂ₚ₊₁ type, with p being an integer between 1 and 12 inclusive;

In particular, R₁ can represent a methyl, ethyl, propyl or butyl radical. Preferably, R₁ represents hydrogen or a methyl radical.
Z is a divalent group chosen from —COO—, —CONH—, —CONCH₃—, —OCO—, —O—, —SO₂—, —CO—O—CO— or —CO—CH₂—CO—; preferably, Z is chosen from COO and CONH.
x is 0 or 1, preferably 1.
R₂ and R₃ are, independently of one another, a saturated or unsaturated, optionally aromatic, linear, branched or cyclic, divalent carbon-comprising radical of 1 to 30 carbon atoms which can comprise from 1 to 18 heteroatoms chosen from O, N, S, F, Si and P;

In the R₂ and/or R₃ radicals, the heteroatom or heteroatoms, when they are present, can be inserted in the chain of the said R₂/R₃ radical or else the said R₂/R₃ radical can be substituted by one or more groups comprising them, such as hydroxyl or amino (NH₂, NHR′ or NR′R″, with R′ and R″, which are identical or different, representing a linear or branched C₁-C₂₂ alkyl, in particular methyl or ethyl).

In particular, R₂ and/or R₃ can be:
an alkylene radical having from 1 to 20 carbon atoms, such as methylene, ethylene, n-propylene, isopropylene, n-butylene, isobutylene, tert-butylene, pentylene, isopentylene, n-hexylene, isohexylene, heptylene, isoheptylene, n-octylene, iso-octylene, nonylene, isononylene, decylene, isodecylene, n-dodecylene, isododecylene, tridecylene, n-tetradecylene, hexadecylene, n-octadecylene, docosylene or arachidylene;
a substituted or unsubstituted cycloalkylene radical having from 5 to 10 carbon atoms, such as cyclopentylene, cyclohexylene, cycloheptylene, cyclooctylene, cyclononylene or cyclodecylene;
a phenylene —C₆H₄— radical (ortho, meta or para) optionally substituted by a C₁-C₁₂ alkyl radical optionally comprising from 1 to 25 heteroatoms chosen from N, O, S, F, Si and/or P; or
a benzylene —C₆H₄—CH₂— radical optionally substituted by a C₁-C₁₂ alkyl radical optionally comprising from 1 to 25 heteroatoms chosen from O, N, S, F, Si and P;
or a mixture of these radicals;
m is 0 or 1; preferably 1;
n is between 1 and 100, preferably 1 and 5, inclusive;
X is:

(a) a guanidino group or an amidino group; or

(b) a group of formula —N(R₆)(R₇) with R₆ and R₇ representing, independently of one another, (i) a hydrogen atom, (ii) a saturated or unsaturated, optionally aromatic, linear, branched or cyclic alkyl group comprising from 1 to 18 carbon atoms, in particular from 1 to 6 carbon atoms, which can comprise from 1 to 10 heteroatoms chosen from O, N, S, F, Si and P; (iii) it being possible for R₆ and R₇ to form, with the nitrogen atom, a saturated or unsaturated, optionally aromatic, ring of formula:

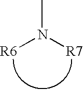

comprising in total from 5 to 8 atoms and in particular from 3 to 7 carbon atoms and/or from 1 to 4 heteroatoms chosen from O, S and N; it being possible for the said first ring to be fused to one or more other saturated or unsaturated, optionally aromatic, rings each comprising from 5 to 7 atoms and in particular from 4 to 7 carbon atoms and/or from 2 to 4 heteroatoms chosen from O, S and N.

For example, R₆ and R₇ can be chosen from hydrogen or a methyl, ethyl, propyl, isopropyl, n-butyl, t-butyl, isobutyl, octyl, lauryl or stearyl group.

Preferably, R₆ and R₇ are chosen, independently of one another, from H, CH₃ and C₂H₅.

(c) a ring:

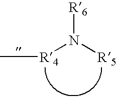

in which R′₄ and R′₅ form, with the nitrogen atom, a saturated or unsaturated, optionally aromatic, ring comprising in total from 5 to 8 atoms and in particular from 3 to 7 carbon atoms and/or from 1 to 4 heteroatoms chosen from O, S and N; it being possible for the said ring to be fused to one or more other saturated or unsaturated, optionally aromatic, rings each comprising from 5 to 7 atoms and in particular from 4 to 7 carbon atoms and/or from 2 to 4 heteroatoms chosen from O, S and N; and R′₆ is chosen from H, —CH₃ and —C₂H₅.

For example, X can constitute an aromatic or non-aromatic ring comprising a tertiary amine group or can represent an aromatic or nonaromatic heterocycle comprising a tertiary nitrogen. Mention may be made of the radicals of pyridinyl, indolyl, isoindolinyl, imidazolyl, imidazolinyl, piperidinyl, piperazinyl, pyrazolyl, pyrazolinyl, quinolinyl, pyrrolidinyl, quinidinyl or morpholinyl type and their mixtures.

Preferably, X is:
a group of formula —N(R₆)(R₇) with R₆ and R₇ representing, independently of one another, (i) a hydrogen atom, (ii) a saturated or unsaturated, optionally aromatic, linear, branched or cyclic alkyl group comprising from 1 to 18 carbon atoms, in particular from 1 to 6 carbon atoms, (iii) it being possible for R₆ and R₇ to form, with the nitrogen atom, a saturated or unsaturated, optionally aromatic, ring of formula:

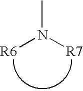

comprising in total from 5 to 7 atoms and in particular from 4 to 6 carbon atoms and optionally from 1 to 3 heteroatoms chosen from O, S and N;
a ring:

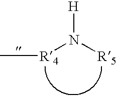

in which R′₄ and R′₅ form, with the nitrogen atom, a saturated or unsaturated, optionally aromatic, ring comprising from 5 to 7 atoms and in particular from 4 to 6 carbon atoms.

Preferably, X is an NH₂, N(CH₃)₂, pyridinyl, imidazolyl, piperidinyl, piperazinyl or morpholinyl group.
X′⁺ is a divalent group of formula —N⁺(R₆)(R₇)— with R₆ and R₇ representing, independently of one another, (i) a hydrogen atom, (ii) an optionally aromatic, linear, branched or cyclic alkyl group comprising from 1 to 25 carbon atoms which can comprise from 1 to 20 heteroatoms chosen from O, N, S and P; (iii) it being possible for R₆ and R₇ to form, with the nitrogen atom, a saturated or unsaturated, optionally aromatic, ring of formula:

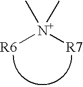

comprising in total from 5 to 8 atoms and in particular from 3 to 7 carbon atoms and/or from 1 to 4 heteroatoms chosen from O, S and N; it being possible for the said first ring to be fused to one or more other saturated or unsaturated, optionally aromatic, rings each comprising from 5 to 7 atoms and in particular from 4 to 7 carbon atoms and/or from 2 to 4 heteroatoms chosen from O, S and N.

For example, R₆ and R₇ can be chosen from hydrogen or a methyl, ethyl, propyl, isopropyl, n-butyl, t-butyl, isobutyl, octyl, lauryl or stearyl group. Preferably, R₆ and R₇ are chosen, independently of one another, from H, CH₃ and C₂H₅.
X′+ can also be a divalent ring of formula:

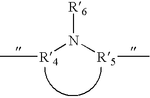

in which R′₄ and R′₅ form, with the nitrogen atom, a saturated or unsaturated, optionally aromatic, ring comprising in total from 5 to 8 atoms and in particular from 3 to 7 carbon atoms and/or from 1 to 4 heteroatoms chosen from O, S and N; it being possible for the said ring to be fused to one or more other saturated or unsaturated, optionally aromatic, rings each comprising from 5 to 7 atoms and in particular from 4 to 7 carbon atoms and/or from 2 to 4 heteroatoms chosen from O, S and N; and R′₆ is chosen from H, —CH₃ and —C₂H₅.

Mention may be made, among the preferred X′⁺ radicals, of divalent radicals of pyridinyl, imidazolyl, piperidinyl, piperazinyl, pyrrolidinyl or morpholinyl type, and their mixtures; and more particularly of pyridinyl, imidazolyl and piperazinyl divalent radicals.
Y′⁻ is a group chosen from —COO⁻, —SO₃⁻, —OSO₃⁻, —PO₃²⁻ and —OPO₃²⁻.

Mention may be made, among the preferred monomers of formula (I), alone or as a mixture, of:
dimethylaminopropyl(meth)acrylamide (−0.343 and 0.210), dimethylaminoethyl(meth)acrylamide (−0.250 and 0.302) or diethylaminopropyl(meth)acrylamide (0.813);
diethylaminoethyl (meth)acrylate, dimethylaminoethyl (meth)acrylate (0.948 and 1.5), morpholinoethyl (meth)acrylate (0.554) or tert-butylaminoethyl (meth)acrylate;
vinylimidazole (0.96), vinylpyridine (1.20), vinylamine,

and the following monomers:

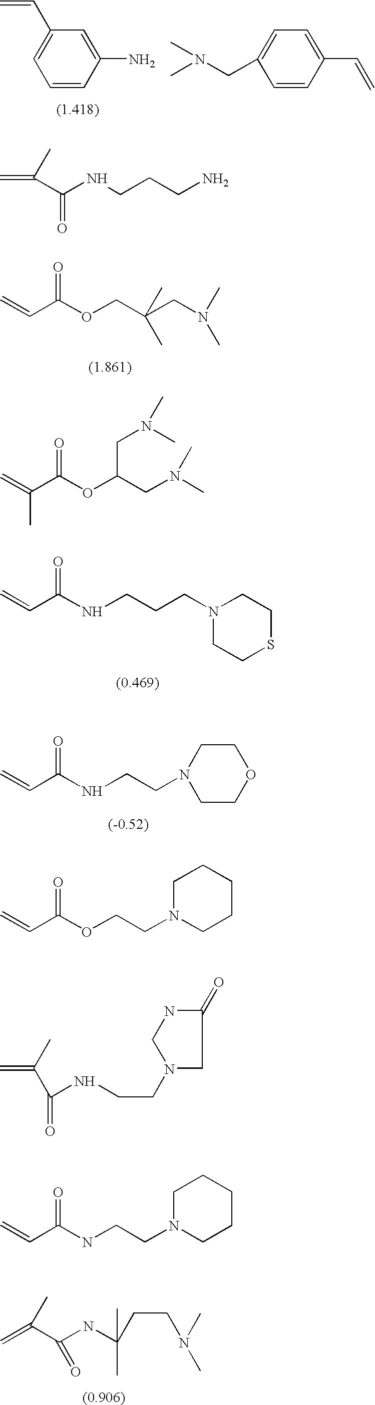

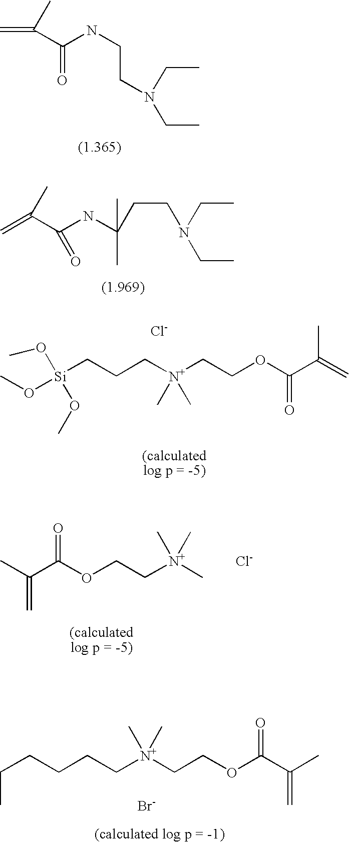

Mention may be made, among the monomers of formula (II), of N,N-dimethyl-N-(2-methacryloyloxyethyl)-N-(3-sulphopropyl)ammonium betaine (in particular SPE from Raschig); N,N-dimethyl-N-(3-methacrylamidopropyl)-N-(3-sulphopropyl)ammonium betaine (SPP from Raschig) and 1-(3-sulphopropyl)-2-vinylpyridinium betaine (SPV from Raschig), and also 2-methacryloyloxyethyl phosphorylcholine.

Mention may be made, among the very particularly preferred hydrophilic cationic monomers capable of being used, of the following monomers:
dimethylaminopropyl(meth)acrylamide or dimethylaminoethyl(meth)acrylamide,
diethylaminoethyl (meth)acrylate, dimethylaminoethyl (meth)acrylate or morpholinoethyl (meth)acrylate;
vinylimidazole or vinylpyridine,

and the following monomers:

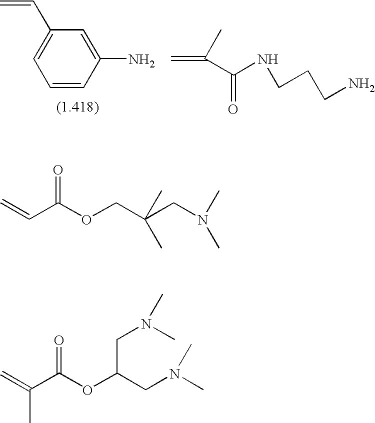

The amine units can be completely or partially neutralized by carboxylic acids preferably having a linear or branched alkyl chain having at least 6 carbon atoms, in particular from 6 to 32 carbon atoms, indeed even from 12 to 24 carbon atoms.

The carboxylic acids can additionally comprise one or more heteroatoms chosen from O and N, for example in the form of hydroxyl groups. Mention may in particular be made of acetic acid, α-hydroxyethanoic acid, α-hydroxyoctanoic acid, α-hydroxycaprylic acid, ascorbic acid, benzoic acid, behenic acid, capric acid, caproic acid, caprylic acid, citric acid, dodecylbenzenesulphonic acid, 2-ethylcaproic acid, folic acid, fumaric acid, galactaric acid, gluconic acid, glycolic acid, 2-hexadecyleicosanoic acid, hydroxycaproic acid, 12-hydroxystearic acid, isolauric (or 2-butyloctanoic) acid, isomyristic (or 2-hexyloctanoic) acid, isoarachidic (or 2-octyldodecanoic) acid, isolignoceric (or 2-decyltetradecanoic) acid, lactic acid, lauric acid, malic acid, myristic acid, oleic acid, palmitic acid, propionic acid, sebacic acid, stearic acid, tartaric acid, terephthalic acid, trimesic acid, undecylenic acid and their mixtures.

Neutralization can be carried out before, during or after the polymerization reaction. Preferably, neutralization is carried out during the polymerization.

The amine units of the polymer can also be completely or partially quaternized. Quaternization can be carried out before or after the polymerization reaction, using quaternization agents known for carrying out such reactions, such as, for example, halogenated derivatives (for example, alkyl halide, in particular C₆-C₃₂ alkyl halide, in particular octyl bromide).

The ethylenic polymer according to the invention can comprise a single hydrophilic cationic monomer or a mixture of such hydrophilic cationic monomers.

Thus, in a first embodiment of the invention, the polymers present in the dispersion according to the invention can result solely from the polymerization of one or more hydrophilic cationic monomers, which will thus represent 100% by weight of the total weight of starting monomers.

However, according to a second embodiment of the invention, the polymers present in the dispersion can result from the polymerization of one or more hydrophilic cationic monomers and of one or more additional monomers, which will thus have a log p of greater than 2, and/or be noncationic, and which can thus be present in a proportion of 0.01 to 50% by weight, in particular of 0.5 to 49% by weight, indeed even of 1 to 48% by weight, better still from 2 to 45% by weight, preferably from 5 to 40% by weight and also from 15 to 35% by weight, with respect to the total weight of monomers.

In this case, the hydrophilic cationic monomers represent from 50% to 99.99% by weight, indeed even from 51% to 99.5% by weight, especially from 52% to 99% by weight, better still from 55% to 98% by weight, preferably from 60% to 95% by weight and also from 65% to 85% by weight, with respect to the total weight of monomers.

The additional monomers can in particular be chosen from the following monomers, alone or as a mixture:
(i) (meth)acrylic acid esters of formula CH₂═CHCOOR₄ or CH₂═C(CH₃)COOR₄ with R₄ representing a saturated or unsaturated, linear, cyclic or branched carbon-comprising chain, in particular hydrocarbon (alkyl) chain, having from 1 to 32 carbon atoms and optionally comprising, inserted, one or more heteroatoms chosen from O, N, S and/or optionally substituted by one or more substituents chosen from —OH and halogen atoms (Cl, Br, I and F).

In particular, the carbon-comprising chain R₄ can be:
a C₁-C₃₂ alkyl group;
a C₃ to C₈ cycloalkyl group;
a C₆ to C₂₀ aryl group;
a C₇ to C₃₀ aralkyl group (C₁ to C₄ alkyl group);
a 4- to 12-membered heterocyclic group comprising one or more heteroatoms chosen from O, N and S;
a heterocycloalkyl group (C₁ to C₄ alkyl group), such as a furfuryl group;
   it being possible for the said alkyl, cycloalkyl, aryl, aralkyl, heterocyclic or heterocycloalkyl groups to comprise, inserted, one or more heteroatoms chosen from O, N and S and/or to be substituted by one or more substituents chosen from the hydroxyl group, halogen atoms and linear or branched C₁-C₄ alkyl groups in which one or more heteroatoms chosen from O, N, S and P is/are optionally inserted, it being possible for the said alkyl groups in addition to be optionally substituted by one or more substituents chosen from the hydroxyl group and halogen atoms (Cl, Br, I and F).

Mention may thus be made of methyl, ethyl, propyl, n-butyl, isobutyl, 2-ethylhexyl, octyl, isooctyl, isodecyl, decyl, lauryl, tridecyl, dodecyl, myristyl, cetyl, palmityl, stearyl, behenyl, oleyl; tert-butyl, t-butylcyclohexyl, t-butylbenzyl, furfuryl and isobornyl (meth)acrylates; 2-hydroxyethyl (meth)acrylate, ethoxyethyl (meth)acrylate; 2-methoxyethyl acrylate and hydroxypropyl acrylate.
(ii) (meth)acrylamides of formula CH₂═CHCONR₅R′₅ or CH₂═C(CH₃)CONR₅R′₅ in which R₅ and R′₅, which are identical or different, represent a hydrogen atom or a saturated or unsaturated, optionally aromatic, linear, cyclic or branched carbon-comprising chain, in particular a hydrocarbon (alkyl) chain, having from 6 to 28 carbon atoms and optionally comprising, inserted, one or more heteroatoms chosen from O, N and S and/or optionally substituted by one or more substituents chosen from —OH and halogen atoms (Cl, Br, I and F).

In particular, the carbon-comprising chain R₅ and/or R′₅ can be:
a C₆-C₂₈ alkyl group;
a C₆ to C₈ cycloalkyl group;
a C₅ to C₂₀ aryl group;
a C₅ to C₂₈ aralkyl group (C₁ to C₄ alkyl group);
a 4- to 12-membered heterocyclic group comprising one or more heteroatoms chosen from O, N and S;
a heterocycloalkyl group (C₁ to C₄ alkyl group), such as a furfuryl group;
   it being possible for the said alkyl, cycloalkyl, aryl, aralkyl, heterocyclic or heterocycloalkyl groups to comprise, inserted, one or more heteroatoms chosen from O, N and S and/or to be substituted by one or more substituents chosen from the hydroxyl group, halogen atoms and linear or branched C₁-C₄ alkyl groups in which one or more heteroatoms chosen from O, N, S and P is/are optionally inserted, it being possible for the said alkyl groups in addition to be optionally substituted by one or more substituents chosen from the hydroxyl group and halogen atoms (Cl, Br, I and F).

Examples of such monomers are N-(tert-butyl)(meth)acrylamide, N-butyl(meth)acrylamide, N-isobutyl(meth)acrylamide, N-propylmethacrylamide, N-isopropylmethacrylamide, N-hexyl(meth)acrylamide, N-(2-ethylhexyl)(meth)acrylamide, N-octyl(meth)acrylamide, N-isooctyl(meth)acrylamide, N-nonyl(meth)acrylamide, N-undecyl(meth)acrylamide, N-dodecyl(meth)acrylamide, N-tridecyl(meth)acrylamide, N-tetradecyl(meth) acrylamide, N-hexadecyl(meth)acrylamide, N-palmityl(meth)acrylamide, N-octadecyl(meth)acrylamide, N-docosanoyl(meth)acrylamide, N-octadecenoyl(meth) acrylamide, N-cyclohexyl(meth)acrylamide, N-phenyl(meth)acrylamide, N-isobornyl(meth)acrylamide, N-benzyl(meth)acrylamide or N,N-dibutyl(meth)acrylamide.
(iii) vinyl esters of formula CH₂═CH—OCO—R₆ with R₆ representing a saturated or unsaturated, linear or branched carbon-comprising chain, in particular a hydrocarbon chain, having from 1 to 12 carbon atoms, among which may be mentioned vinyl acetate, vinyl butyrate (or butanoate), vinyl ethylhexanoate, vinyl neononanoate, vinyl neododecanoate, vinyl propionate, vinyl hexanoate, vinyl ethylhexanoate, vinyl octanoate, vinyl decanoate, vinyl pivalate, vinyl palmitate, vinyl stearate, vinyl cyclohexanoate, vinyl benzoate or vinyl 4-(tert-butyl)benzoate;
(iv) vinyl ethers of formula CH₂═CHOR₇ with R₇ representing a saturated or unsaturated, linear or branched carbon-comprising chain, in particular a hydrocarbon chain, having from 1 to 12 carbon atoms, among which may be mentioned ethyl vinyl ether, ethylhexyl vinyl ether, butyl vinyl ether, isobutyl vinyl ether, cyclohexyl vinyl ether, octyl vinyl ether, decyl vinyl ether, dodecyl vinyl ether, hexadecyl vinyl ether and octadecyl vinyl ether.
(v) vinyl compounds of formula CH₂═CHR₈ in which R₈ is
a hydroxyl group;
a linear or branched alkyl group comprising from 1 to 25 carbon atoms in which one or more heteroatoms chosen from O, N, S and P is/are optionally inserted; it being possible for the said alkyl group in addition to be optionally substituted by one or more substituents chosen from —OH and halogen atoms (Cl, Br, I and F);
a C₃ to C₈ cycloalkyl group, such as cyclohexyl,
a C₆ to C₂₀ aryl group, such as phenyl,
a C₇ to C₃₀ aralkyl group (C₁ to C₄ alkyl group), such as 2-phenylethyl or benzyl,
a 4- to 12-membered heterocyclic group comprising one or more heteroatoms chosen from O, N and S,
a heterocycloalkyl group (alkyl group of 1 to 4 carbons), such as furfuryl, furfurylmethyl or tetrahydrofurfurylmethyl,
   it being possible for the said cycloalkyl, aryl, aralkyl, heterocyclic or heterocycloalkyl groups to be optionally substituted by one or more substituents chosen from the hydroxyl group, halogen atoms and linear or branched C₁-C₄ alkyl groups in which one or more heteroatoms chosen from O, N, S and P is/are optionally inserted, it being possible for the said alkyl groups in addition to be optionally substituted by one or more substituents chosen from —OH and halogen atoms (Cl, Br, I and F).

Examples of vinyl monomers are vinylcyclohexane, styrene, vinylcaprolactam, methylstyrene, 4-(tert-butyl)styrene, 4-acetoxystyrene, 4-methoxystyrene, 3-methylstyrene, 4-methylstyrene, 2-chlorostyrene, 3-chlorostyrene, 4-chlorostyrene, 2,6-dichlorostyrene, 2,4-dimethylstyrene, 2,5-dimethylstyrene, 3,5-ethoxystyrene, 2,4-ethoxystyrene, vinylbutyral, vinyl chloride, vinylformal, vinylidene chloride, vinylidene fluoride, 2-vinylnaphthalene, N-methylmaleimide, 1-octene, 1-butene, chlorobutadiene, chlorotrifluoroethylene; cis-isoprene, trans-isoprene, 1-octadecene, butadiene, hexadecene, eicosene or 4-fluorostyrene.
(vi) multifunctional monomers, in particular difunctional monomers, such as di(meth)acrylates or tri(meth)acrylates, chosen in particular from 1,4-butanediol dimethacrylate, 1-6-hexanediol dimethacrylate, 1,12-dodecanediol dimethacrylate, diallyl phthalate, divinylbenzene, poly(ethylene glycol) dimethacrylate and their mixtures.
(vii) (meth)acrylic acid, maleic anhydride, crotonic acid, itaconic acid, fumaric acid, maleic acid, diacrylic acid, dimethylfumaric acid, citraconic acid, acrylamidopropanesulphonic acid, 2-acrylamido-2-methylpropanesulphonic acid, styrenesulphonic acid, vinylbenzoic acid, vinylphosphoric acid, vinylsulphonic acid, vinylbenzenesulphonic acid, acrylamidoglycolic acid of formula CH₂═CH—CONHCH(OH)COOH, vinylphosphonic acid, sulphopropyl (meth)acrylate, sulphoethyl (meth)acrylate, vinyl methyl sulphone, 2-(methacryloyloxy)ethyl phosphate of formula CH₂═C(CH₃)COOC₂H₄OP(O)(OH)₂, and their salts and their mixtures.
(viii) poly(ethylene glycol) (meth)acrylates, alkyl poly(ethylene glycol) (meth)acrylates and more particularly methyl poly(ethylene glycol) methacrylates.

Preferably, the additional monomers can be chosen from (meth)acrylic acid C₁-C₁₈ esters, in particular from methyl, ethyl, propyl, n-butyl, isobutyl, 2-ethylhexyl, tert-butyl, isooctyl, decyl, myristyl or stearyl (meth)acrylates, (meth)acrylamides, in particular from methyl-, ethyl-, propyl-, (n-butyl)-, isobutyl-, (2-ethylhexyl)-, (tert-butyl)-, isooctyl-, decyl-, myristyl- or stearyl(meth)acrylamides, styrene, ethylhexyl vinyl ether, dodecyl vinyl ether, vinyl hexanoate; 2-hydroxyethyl acrylate and poly(ethylene glycol) methacrylate, and their mixtures.

Preferably, the polymer according to the invention exhibits a glass transition temperature (Tg) of less than or equal to 30° C., in particular of between −150° C. and 25° C., in particular between −70° C. and 20° C., preferably between −50° C. and 0° C.

In order to bring this about, the polymers present in the dispersion according to the invention can result from the polymerization of one or more monomers with a Tg of less than or equal to 30° C. (“low Tg”), preferably of between −150° C. and 25° C., in particular between −70° C. and 20° C., preferably between −50° C. and 0° C., which can represent 100% by weight of the total weight of starting monomers, or else which can be as a mixture with one or more monomers with a Tg of greater than 30° C. (“high Tg”) but present in an amount such that the overall Tg of the polymer is less than or equal to 30° C. In this second case, the monomer of high Tg, alone or as a mixture, can be present in a proportion of 0.01 to 50% by weight, with respect to the total weight of the monomers, in particular of 0.1 to 40% by weight, indeed even of 1 to 30% by weight or also 5 to 15% by weight; the monomer of low Tg, alone or as a mixture, then being present in a proportion of 50 to 99.99% by weight, in particular of 60 to 99.9% by weight, indeed even from 70 to 99% by weight, or also of 85 to 95% by weight, with respect to the total weight of monomers.

In the present invention, the Tg (or glass transition temperature) values shown are theoretical Tg values determined from the theoretical Tg values of the constitutive monomers of the polymer, which can be found in a reference handbook, such as the Polymer Handbook, 4^{th} ed. (Brandrup, Immergut, Grulke), 1999, John Wiley, according to the following relationship, referred to as the Fox law:

$$\frac{1}{Tg} = {\sum }_{i} ( \frac{ϖ i}{Tgi} )$$

ω̅i being the mass fraction of the monomer i in the polymer and Tgi being the glass transition temperature of the homopolymer of the monomer i (expressed in Kelvin).

In the present description, “Tg monomer” denotes the monomer, the homopolymer of which has such a glass transition temperature.

A person skilled in the art will know how, on the basis of the Fox law and his general knowledge, to determine the amount of each monomer capable of being present in the polymer of the dispersion so as to always obtain, in the end, a dispersion of polymer having a Tg within the desired range.

Preferably, the polymer exhibits only one glass transition temperature.

However, it can exhibit several glass transition temperatures, in particular two Tg values; in this case, preferably, the lowest Tg value is less than 30° C. The monomers with the low Tg value can be chosen from hydrophilic cationic monomers and/or additional monomers; it is the same for the monomers with a high Tg value.

The polymers which can be used in the context of the present invention preferably have a number-average molecular weight (Mn) of between 2000 and 1 000 000, in particular between 3000 and 800 000 and better still between 4000 and 500 000, indeed even between 10 000 and 300 000.

The dispersion of polymer particles according to the invention thus comprises a nonaqueous medium in which the said particles are dispersed.

This nonaqueous medium is composed of at least one nonaqueous compound which is liquid at 25° C. and which has an overall solubility parameter according to the Hansen solubility space of less than or equal to 20 (MPa)^{1/2}, or of a mixture of such compounds.

The overall solubility parameter δ according to the Hansen solubility space is defined in the article “Solubility Parameter Values” by Grulke in the work “Polymer Handbook, 3rd edition, chapter VII, pages 519-559, by the relationship:

δ=(d_{D}²+d_{P}²+d_{H}²)^{1/2}

in which:
d_{D} characterizes the London dispersion forces resulting from the formation of dipoles induced during molecular impacts,
d_{P} characterizes the forces of Debye interactions between permanent dipoles,
d_{H} characterizes the forces of specific interactions (hydrogen bond, acid/base or donor/acceptor type, etc.).

The definition of the solvents in the three-dimensional solubility space according to Hansen is described in the paper by Hansen: “The Three-Dimensional Solubility Parameters”, J. Paint Technol., 39, 105 (1967).

Mention may be made, among nonaqueous liquid compounds having an overall solubility parameter according to the Hansen solubility space of less than or equal to 20 (MPa)^{1/2}, of liquid fatty substances, in particular oils, which can be chosen from optionally branched, carbon-comprising, hydrocarbon, fluorinated and/or silicone, natural or synthetic oils, alone or as a mixture.

Mention may be made, among these oils, of vegetable oils formed by esters of fatty acids and of polyols, in particular triglycerides, such as sunflower, sesame or rapeseed oil, or esters derived from long-chain acids or alcohols (that is to say, having from 6 to 20 carbon atoms), in particular esters of formula RCOOR′ in which R represents the residue of a higher fatty acid comprising from 7 to 19 carbon atoms and R′ represents a hydrocarbon chain comprising from 3 to 20 carbon atoms, such as palmitates, adipates, myristates and benzoates, in particular diisopropyl adipate and isopropyl myristate.

Mention may also be made of hydrocarbons and in particular liquid paraffins, liquid petrolatum, hydrogenated polyisobutylene, C₈-C₁₆ isoparaffins and volatile isoparaffins, such as isododecane or the ‘Isopar’ products.

Mention may also be made of silicone oils, such as polydimethylsiloxanes and polymethylphenylsiloxanes, optionally substituted by optionally fluorinated aliphatic and/or aromatic groups or by functional groups such as hydroxyl, thiol and/or amine groups, and volatile silicone oils, in particular cyclic or linear volatile silicone oils, such as cyclodimethylsiloxanes, cyclophenylmethylsiloxanes and linear dimethylsiloxanes, among which may be mentioned linear dodecamethylpentasiloxane (L5), octamethylcyclotetrasiloxane, decamethylcyclopentasiloxane, hexadecamethylcyclohexasiloxane, heptamethylhexyltrisiloxane or heptamethyloctyltrisiloxane.

Mention may also be made of solvents, alone or as a mixture, chosen from linear, branched or cyclic esters having from 6 to 30 carbon atoms, ethers having from 6 to 30 carbon atoms and ketones having from 6 to 30 carbon atoms.

Mention may also be made, among the nonaqueous compounds capable of being employed, of monoalcohols having an overall solubility parameter according to the Hansen solubility space of less than or equal to 20 (MPa)^{1/2}, that is to say fatty aliphatic monoalcohols having at least 6 carbon atoms, in particular from 6 to 32, the hydrocarbon chain not comprising a substituent group. Mention may be made, as monoalcohols according to the invention, of oleyl alcohol, decanol, dodecanol, octadecanol and linoleyl alcohol.

Preferably, the nonaqueous medium comprises volatile silicone oils, in particular cyclic or linear volatile silicone oils, such as cyclodimethylsiloxanes and linear dimethylsiloxanes, and/or esters of formula RCOOR′ in which R represents the residue of a higher fatty acid comprising from 7 to 19 carbon atoms and R′ represents a hydrocarbon chain comprising from 3 to 20 carbon atoms, such as palmitates, adipates, myristates and benzoates, in particular diisopropyl adipate and isopropyl myristate, and their mixtures.

The choice of the nonaqueous medium can be easily made by a person skilled in the art according to the nature of the monomers constituting the polymer and/or the nature of the stabilizing agent.

The polymer dispersion can be manufactured as described in the document EP-A-749 747. Generally, the polymerization can be carried out in dispersion, that is to say by precipitation of the polymer during formation, with protection of the particles formed using a stabilizing agent.

It is thus possible to prepare a mixture comprising the starting monomers and a radical initiator. This mixture is dissolved in a synthesis solvent.

The monomers are soluble in the reaction medium, whereas the polymer is insoluble therein. As the polymerization proceeds, the polymer will precipitate and will be stabilized by the stabilizing agent present. Polymer particles protected at the surface by the stabilizing agent are thus obtained.

The polymerization can be carried out directly in the nonaqueous medium, which can thus also act as synthesis solvent.

The polymerization can also be carried out in a synthesis solvent and then a solvent exchange can subsequently be carried out, the synthesis solvent being replaced by the nonaqueous medium.

Thus, when the nonaqueous medium chosen is a nonvolatile hydrocarbon or silicone oil, the polymerization can be carried out in a nonpolar organic solvent (synthesis solvent), then the nonvolatile oil (which has to be miscible with the said synthesis solvent) can be added and the synthesis solvent can be selectively distilled.

A synthesis solvent is thus preferably chosen such that the starting monomers and the radical initiator are soluble therein and the particles of polymer obtained are insoluble therein in order for them to precipitate therefrom during their formation. In particular, the synthesis solvent can be chosen from alkanes, such as heptane, isododecane or cyclohexane.

When the nonaqueous medium chosen is a volatile hydrocarbon or silicone oil, the polymerization can be carried out directly in the said oil, which thus also acts as synthesis solvent. The monomers should preferably also be soluble therein, as also the radical initiator, and the polymer obtained should be insoluble therein.

The monomers are preferably present in the synthesis solvent, before polymerization, in a proportion of 5-80% by weight. All the monomers may be present in the solvent before the beginning of the reaction or a portion of the monomers may be added as the polymerization reaction progresses.

The radical initiator can, for example, be an azo or peroxide compound, such as azobisisobutyronitrile or tert-butyl peroxy(2-ethylhexanoate).

The polymer particles are stabilized at the surface.

In a first embodiment, the particles can be stabilized at the surface as the polymerization occurs, by virtue of a stabilizing agent which can in particular be a sequential polymer, a grafted polymer and/or a random polymer, alone or as a blend. Stabilization can be carried out by any known means and in particular by polymerization in the presence of the stabilizing agent.

Preferably, the stabilizing agent is present in the mixture at the start of the polymerization. However, it is also possible to add it continuously, in particular when monomers are also being added continuously.

In a second embodiment, the polymer can be synthesized in a synthesis solvent and then dispersed in a nonaqueous dispersing medium by addition of the dispersing agent, and the synthesis solvent is evaporated.

Dispersing agent is understood to mean a polymer not covalently bonded to the ethylenic polymer defined above.

Use may be made of 0.1 to 30% by weight of stabilizing agent, with respect to the weight of the starting mixture of monomers, and preferably of 1 to 20% by weight, preferably of 2 to 15% by weight, indeed even of 3 to 10% by weight.

When a grafted and/or sequential polymer is used as stabilizing agent, the synthesis solvent is chosen so that at least a portion of the grafts or sequences of the said stabilizing polymer is soluble in the said solvent, the other portion of the grafts or sequences being insoluble therein. The stabilizing polymer used during the polymerization must be soluble or dispersible in the synthesis solvent. Furthermore, the choice is preferably made of a stabilizing agent comprising a portion (sequences, grafts or others) exhibiting a degree of affinity for the polymer formed during the polymerization.

When a random polymer is used as stabilizing agent, it is chosen so that it has a sufficient amount of groups rendering it soluble in the synthesis solvent envisaged.

Mention may be made, among the grafted polymers, of silicone polymers grafted with a hydrocarbon chain and hydrocarbon polymers grafted with a silicone chain.

Grafted copolymers having, for example, an insoluble backbone of polyacrylic type with soluble grafts of poly(12-hydroxystearic acid) type are also suitable.

Mention may also be made, as stabilizing polymer, of:
(a) grafted or sequential block copolymers comprising at least one block of polyorganosiloxane type and at least one block of a polymer (i) resulting from radical polymerization or (ii) resulting from polycondensation, in particular of polyether, polyester or polyamide type, or their blend, it being possible for the said copolymer to comprise fluorinated entities.

Mention may be made, as grafted or sequential block copolymers comprising at least one block of polyorganosiloxane type and at least one block of a radical polymer, of grafted copolymers of acrylic/silicone type which can be employed in particular when the nonaqueous medium is a silicone medium.

When the grafted or sequential block copolymers comprise at least one block of polyorganosiloxane type and at least one polyether block, the polyorganosiloxane block can in particular be a polydimethylsiloxane or alternatively a poly(C₂-C₁₈)alkylmethylsiloxane; the polyether block can be a polyoxy(C₂-C₁₈)alkylene, in particular polyoxyethylene and/or polyoxypropylene. Use may thus be made of dimethicone copolyols or also (C₂-C₁₈) alkyl methicone copolyols which are optionally crosslinked. Use may be made, for example, of the dimethicone copolyols (silicone polyethers) sold under the name “DC3225C” or “DC5225C” by Dow Corning or the lauryl methicone copolyol sold under the name “DC Q2-5200” by Dow Corning.

Mention may also be made of lauryl dimethicone copolyol crosspolymer, for example KSG31 or KSG32 from Shin-Etsu, cetyl dimethicone copolyol, such as DMC 3071 from GE, and dimethicone copolyol PPG-3 oleyl ether, such as KF-6026 from Shin-Etsu, and also dimethicone copolyol PEG-10 dimethicone, sold by Shin-Etsu under the name KF-6017.
(b) grafted or sequential block copolymers of C₁-C₄ alkyl (meth)acrylates and of C₈-C₃₀ alkyl (meth)acrylates. Mention may be made of the stearyl methacrylate/methyl methacrylate copolymer.
(c) grafted or sequential block copolymers comprising at least one block resulting from the polymerization of ethylenic monomer, comprising one or more optionally conjugated ethylenic bonds, and/or in particular of dienes; and at least one block of polymer resulting from radical polymerization other than dienes, in particular resulting from vinyl, (meth)acrylic or (meth)acrylamide monomer, or of a polyether, of a polyester or of a polyamide, or their blends.

Use may in particular be made of copolymers comprising at least one block resulting from the polymerization of at least one ethylenic monomer, comprising one or more optionally conjugated ethylenic bonds, such as ethylene, butadiene or isoprene, and at least one block of a styrene polymer. When the ethylenic monomer comprises several optionally conjugated ethylenic bonds, the residual ethylenic unsaturations after the polymerization are generally hydrogenated. Thus, in a known way, the polymerization of isoprene results, after hydrogenation, in the formation of an ethylene-propylene block and the polymerization of butadiene results, after hydrogenation, in the formation of an ethylene-butylene block. Mention may be made, among these sequential copolymers, of copolymers of “diblock” or “triblock” type of the polystyrene/polyisoprene or polystyrene/polybutadiene type, such as those sold under the name of “Luvitol HSB” by BASF, of the polystyrene/copoly(ethylene-propylene) type, such as those sold under the name of “Kraton” by Shell Chemical Co., or also of the polystyrene/copoly(ethylene-butylene) type.

Mention may be made, as grafted or sequential block copolymers comprising at least one block resulting from the polymerization of at least one ethylenic monomer, such as ethylene or isobutylene, and at least one block of an acrylic polymer, such as methyl methacrylate, of poly(methyl methacrylate)/polyisobutylene bi- or trisequential copolymers or grafted copolymers comprising a poly(methyl methacrylate) backbone and comprising polyisobutylene grafts.

Mention may be made, as grafted or sequential block copolymers comprising at least one block resulting from the polymerization of at least one ethylenic monomer and at least one block of a polyether, such as a polyoxy(C₂-C₁₈)alkylene, in particular polyoxyethylene and/or polyoxypropylene, of polyoxyethylene/polybutadiene or polyoxyethylene/polyisobutylene bi- or trisequential copolymers.

Use may also be made, as stabilizing agent, of compounds such as:
(d) alkyl dimethicones in which the alkyl group comprises from 6 to 32 carbon atoms, such as lauryl methicone and stearyl methicone, in particular Si tec LDM 3107 from ISP, cetyl dimethicone, such as Abil Wax 9801, behenoxy dimethicone, such as Abil 5440 from Goldschmidt.
(e) dimethiconol esters of formula:

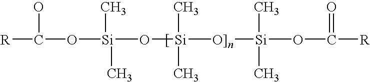

in which R is an alkyl radical having from 6 to 32 carbon atoms, such as dimethiconol behenate, and in particular the products Ultrabee from Noveon and Pecosil DB from Phoenix Chemical.
(f) alkylamidoamines having in particular from 6 to 60 carbon atoms, in particular from 12 to 50 carbon atoms, such as behenamidopropyldimethylamine and in particular Catemol 220 from Phoenix Chemical, of formula:

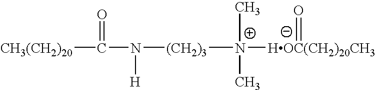

(g) copolymers comprising at least one polyorganosiloxane part and fluorinated groups, and in particular fluorinated silicones or fluorosilicones which can be represented by the formula:

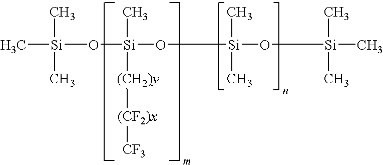

in which x is an integer between 3 and 12, preferably 5 and 10, in particular x=8; y is an integer between 2 and 6, preferably 2 or 3; and m and n are such that the molecular weight of the compound is between 5000 and 15 000;

and more particularly perfluorononyl dimethicones, such as those sold under the names Pecosil FSH-150 and 300 or Pecosil FSL-150 and 300 by Phoenix Chemical.

When the synthesis solvent is nonpolar, it is preferable to choose, as stabilizing agent, a polymer contributing the completest possible covering of the particles, several chains of stabilizing polymers then being adsorbed on one particle of polymer obtained by polymerization.

In this case, it is then preferable to use, as stabilizing agent, either a grafted polymer or a sequential polymer, so as to have better interfacial activity. This is because the sequences or grafts which are insoluble in the synthesis solvent contribute a more voluminous covering at the surface of the particles.

When the liquid synthesis solvent comprises at least one silicone oil, the stabilizing agent is preferably chosen from the group consisting of grafted or sequential block copolymers comprising at least one block of polyorganosiloxane type and at least one block of a radical polymer or of a polyether or of a polyester, such as polyoxypropylenated and/or polyoxyethylenated blocks.

Particularly preferably, the dispersion according to the invention is such that:

1) the hydrophilic cationic monomers are chosen from:
dimethylaminopropyl(meth)acrylamide or dimethylaminoethyl(meth)acrylamide,
diethylaminoethyl (meth)acrylate, dimethylaminoethyl (meth)acrylate or morpholinoethyl (meth)acrylate;
vinylimidazole or vinylpyridine,

and the following monomers:

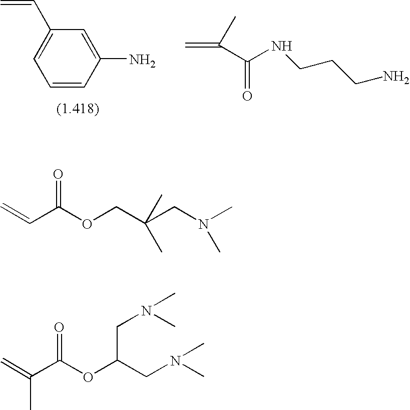

and/or

2) the stabilizing agent is chosen from:
fluorinated silicones or fluorosilicones of formula:

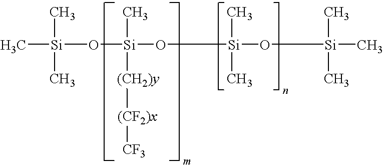

in which x=8, y=2 or 3 and m and n are such that the molecular weight of the compound is between 5000 and 15 000; and
grafted or sequential block copolymers comprising at least one block of polyorganosiloxane type and at least one block of a polymer resulting from polycondensation, in particular of polyether, polyester or polyamide type; especially a polyether block of polyoxy(C₂-C₁₈)alkylene type, in particular polyoxyethylene and/or polyoxypropylene type,

and/or

3) the nonaqueous liquid compound is chosen from volatile silicone oils, in particular cyclic or linear volatile silicone oils, such as cyclodimethylsiloxanes and linear dimethylsiloxanes, and/or esters of formula RCOOR′ in which R represents the residue of a higher fatty acid comprising from 7 to 19 carbon atoms and R′ represents a hydrocarbon chain comprising from 3 to 20 carbon atoms, such as palmitates, adipates, myristates and benzoates, in particular diisopropyl adipate and isopropyl myristate; and their mixtures.

It is possible to add a plasticizer to the dispersion of polymers, so as to lower the Tg of the polymers used. The plasticizer can be chosen from the plasticizers conventionally used in the field of application and in particular from the compounds capable of being solvents for the polymer. The plasticizer can be incorporated during the synthesis or added once the synthesis has been carried out.

The dispersions obtained according to the invention can then be used in a composition, in particular a cosmetic or pharmaceutical composition, which furthermore comprises a cosmetically or pharmaceutically acceptable medium.

Depending on the application envisaged, use may be made of dispersions of film-forming or non-film-forming polymers in a nonaqueous medium comprising volatile or nonvolatile oils.

The composition can then comprise, depending on the application envisaged, the constituents standard to this type of composition.

Mention may be made, among these constituents, of waxes, oils, gums and/or pasty fatty substances of vegetable, animal, mineral or synthetic, indeed even silicone, origin, and their mixtures.

Mention may be made, among the waxes capable of being present in the composition according to the invention, alone or as a mixture, of hydrocarbon waxes, such as beeswax; carnauba, candelilla, ouricury or Japan wax, cork fibre or sugarcane waxes; paraffin or lignite waxes; microcrystalline waxes; lanolin wax; montan wax; ozokerites; polyethylene waxes; waxes obtained by the Fischer-Tropsch synthesis; oils which are hydrogenated, fatty esters and glycerides which are solid at 25° C. Use may also be made of silicone waxes, among which may be mentioned alkyl derivatives, alkoxy derivatives and/or ester derivatives of polymethylsiloxane.

The composition according to the invention can also comprise carbon-comprising, hydrocarbon, fluorinated and/or silicone oils of mineral, animal, vegetable or synthetic origin, alone or as a mixture, in so far as they form a homogeneous and stable mixture and in so far as they are compatible with the use envisaged. Mention may be made, among the oils capable of being present in the composition according to the invention, alone or as a mixture, of hydrocarbon oils, such as liquid paraffin or liquid petrolatum; perhydrosqualene; arara oil; sweet almond, calophyllum, palm, castor, avocado, jojoba, olive or cereal germ oil; esters of lanolic acid, of oleic acid, of lauric acid, of stearic acid; alcohols, such as oleyl alcohol, linoleyl or linolenyl alcohol, isostearyl alcohol or octyldodecanol. Mention may also be made of silicone oils, such as PDMSs, which are optionally phenylated, such as phenyl trimethicones. Use may also be made of volatile oils, such as cyclotetradimethylsiloxane, cyclopentadimethylsiloxane, cyclohexadimethylsiloxane, methylhexyldimethylsiloxane, hexamethyldisiloxane or isoparaffins.

The composition according to the invention can also comprise one or more colouring materials chosen from pulverulent compounds and/or fat-soluble or water-soluble dyes, for example in a proportion of 0.01 to 70% of the total weight of the composition.

The pulverulent compounds can be chosen from the pigments and/or pearlescent agents and/or fillers commonly used in cosmetic or pharmaceutical compositions. Advantageously, the pulverulent compounds represent from 0.1 to 50% of the total weight of the composition and better still from 1 to 40%.

The pigments can be white or coloured, inorganic and/or organic and interferential or noninterferential. Mention may be made, among inorganic pigments, of titanium dioxide, optionally treated at the surface, zirconium or cerium oxides, and also iron or chromium oxides, manganese violet, ultramarine blue, chromium hydrate and ferric blue. Mention may be made, among organic pigments, of carbon black, pigments of D&C type and lakes based on cochineal carmine of barium, strontium, calcium or aluminium.

The pearlescent pigments can be chosen from white pearlescent pigments, such as mica covered with titanium oxide or with bismuth oxychloride, coloured pearlescent pigments, such as titanium oxide-coated mica with iron oxides, titanium oxide-coated mica with in particular ferric blue or chromium oxide, or titanium oxide-coated mica with an organic pigment of the abovementioned type, and also pearlescent pigments based on bismuth oxychloride.

The fillers can be inorganic or organic and lamellar or spherical. Mention may be made of talc, mica, silica, kaolin, Nylon and polyethylene, poly-β-alanine and polyethylene powders, Teflon, lauroyl-lysine, starch, boron nitride, tetrafluoroethylene polymer powders, hollow microspheres, such as Expancel (Nobel Industrie) or Polytrap (Dow Corning), and silicone resin microbeads (Tospearls from Toshiba, for example), precipitated calcium carbonate, magnesium carbonate, basic magnesium carbonate, hydroxyapatite, hollow silica microspheres (Silica Beads from Maprecos), glass or ceramic microcapsules, metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

The fat-soluble dyes are, for example, Sudan red, D&C Red 17, D&C Green 6, β-carotene, soybean oil, Sudan brown, D&C Yellow 11, D&C Violet 2, D&C Orange 5 or quinoline yellow. They can represent from 0 to 20% and in particular from 0.01 to 20% of the weight of the composition and better still from 0.1 to 6%. The water-soluble dyes are, for example, beetroot juice or methylene blue and can represent up to 6% of the total weight of the composition.

The composition can also comprise surfactants which can be chosen from cationic, anionic, amphoteric and nonionic surfactants and their mixtures. These surfactants can be present in a proportion of 0.01 to 30% by weight, in particular of 0.05 to 20% by weight, with respect to the total weight of the composition.

The composition can additionally comprise any additive conventionally used in the cosmetics field, such as antioxidants, fragrances, essential oils, preservatives, cosmetic active principles, moisturizing agents, vitamins, essential fatty acids, ceramides, sunscreens, polymers, thickeners or gelling agents. Of course, a person skilled in the art will take care to choose this or these additives and/or their amounts so that the advantageous properties of the composition according to the invention are not, or not substantially, detrimentally affected by the envisaged addition.

The compositions according to the invention can be provided in any form acceptable and conventional for a cosmetic or pharmaceutical composition.

The composition according to the invention can be provided in the form of a suspension, a dispersion, in particular of oil in water, by virtue of vesicles; an optionally thickened, indeed even gelled, oily solution; an oil-in-water, water-in-oil or multiple emulsion; a gel or a foam; an oily or emulsified gel; a dispersion of vesicles, in particular lipid vesicles; a two-phase or multiphase lotion; a spray; of a lotion, of a cream, of an ointment, of a soft paste, of a salve, of a cast or moulded solid, in particular cast or moulded as a stick or in a dish, or also of a compacted solid.

A person skilled in the art can choose the appropriate formulation form, and its method of preparation, on the basis of his general knowledge, taking into account, on the one hand, the nature of the constituents used, in particular their solubility in the vehicle, and, on the other hand, the application envisaged for the composition.

The cosmetic composition according to the invention can be provided in the form of a product for caring for and/or making up the skin of the body or face, the lips and the hair, of an antisun or self-tanning product, indeed even of a hair product.

It has in particular a particularly advantageous application in the hair field, in particular for the form retention of the hairstyle or the shaping of the hair. The hair compositions are preferably shampoos, gels, hairsetting lotions, blow-drying lotions, or fixing and styling compositions, such as lacquers, foams or sprays. The lotions can be packaged in various forms, in particular in vaporizers, pump-action sprays or aerosol containers, in order to provide for application of the composition in the vaporized form or in the foam form.

The compositions in accordance with the invention can be used for washing or treating keratinous substances, such as the hair, skin, eyelashes, eyebrows, nails, lips or scalp and more particularly the hair.

The compositions according to the invention can be detergent compositions, such as shampoos, shower gels and foam baths.

The compositions of the invention can also be provided in the form of rinse-out or leave-in conditioners, of perming, hair straightening, dyeing or bleaching compositions, or else in the form of rinse-out compositions, to be applied before or after a dyeing, bleaching, perming or hair straightening or else between the two stages of a perming or hair straightening.

The compositions of the invention can also be provided in the form of washing compositions for the skin and in particular in the form of solutions or gels for the bath or shower or of make-up removers.

The compositions according to the invention can also be provided in the form of aqueous or aqueous/alcoholic lotions for caring for the skin and/or hair.

The compositions can also be provided in the form of a hair care product (hair mask) or of a lacquer, gel or foam for styling.

Another subject-matter of the invention is a method for the cosmetic treatment of keratinous substances, in particular of the skin of the body or face, the nails, the hair and/or the eyelashes, comprising the application, to the said substances, of a cosmetic composition as defined above.

This method according to the invention makes possible in particular the form retention of the hairstyle or the treatment, care or washing of or removal of make-up from the skin, hair or any other keratinous substance.

The invention is illustrated in more detail in the following examples, in which the Tg value of the polymers is calculated according to the Fox law.

### EXAMPLE 1

The constituents of the vessel heel are introduced into a 500 ml reactor equipped with a reflux condenser, a stirrer and a 250 ml dropping funnel. The mixture is heated to 80° C. in 1 hour and then the constituents of the material to be run in are added in 1 hour. The temperature is maintained at 80° C. for 5 hours.

| | Vessel | | Material to | | Total | |
|---|---|---|---|---|---|---|
| | heel | | be run in | | amount | |
| | (g) | | (g) | | (g) | |
| Dimethylaminoethyl | 10 | | 30 | | 40 | |
| methacrylate (log p = 1.5) | | | | | | |
| Neutralizing agent (palmitic | 17 | | 49 | | 66 | |
| acid) | | | | | | |
| Stabilizing agent: PEG-10 | 2.5 | | 2.5 | | 5 | |
| dimethicone (KF-6017 from | | | | | | |
| Shin-Etsu) | | | | | | |
| Isopropyl myristate | 200 | ml | 100 | ml | 300 | ml |
| Initiator (Trigonox 21S) | 0.3 | | 0.3 | | 0.6 | |

An orange-coloured dispersion of polymer in isopropyl myristate having a dry matter content of 26% is obtained. The hydrophilic cationic monomer represents 100% by weight of the total weight of monomers.

### EXAMPLE 2

The constituents of the vessel heel are introduced into a 500 ml reactor equipped with a reflux condenser, a stirrer and a 250 ml dropping funnel. The mixture is heated to 80° C. in 1 hour and then the constituents of the material to be run in are added in 1 hour. The temperature is maintained at 80° C. for 5 hours.

| | Vessel | | Material to | | Total | |
|---|---|---|---|---|---|---|
| | heel | | be run in | | amount | |
| | (g) | | (g) | | (g) | |
| Dimethylaminoethyl | 5 | | 15 | | 20 | |
| methacrylate (log p = 1.5) | | | | | | |
| 2-Ethylhexyl acrylate (log | 5 | | 10 | | 15 | |
| p = 4.33) | | | | | | |
| Neutralizing agent (palmitic | 8.5 | | 24.5 | | 33 | |
| acid) | | | | | | |
| Stabilizing agent* | 2.5 | | 2.5 | | 5 | |
| Isopropyl myristate | 200 | ml | 150 | ml | 350 | ml |
| Initiator (Trigonox 21S) | 0.5 | | 0.5 | | 1 | |

*Stabilizing agent: Cyclopentasiloxane (and) PEG/PPG-18/18 Dimethicone (DC5225C from Dow Corning)

An orange-coloured dispersion of polymer in isopropyl myristate having a dry matter content of 20% is obtained. The hydrophilic cationic monomer represents 57% by weight of the total weight of monomers.

### EXAMPLE 3

The following compositions are prepared:

| | Composition | |
|---|---|---|
| | A | Composition B |
| Dispersion of Example 1 | 1.0% AM of | — |
| | polymer | |
| Dispersion of Example 2 | — | 1.0% AM of |
| | | polymer |
| Dimethicone copolyol/D5 | 0.5% AM | 0.5% AM |
| (DC5225C) | dimethicone | dimethicone |
| | copolyol | copolyol |
| Cyclopentasiloxane (D5) | 10.0% | 10.0% |
| Trimethylbehenylammonium | 1.2% | 1.2% |
| chloride | | |
| Propylene glycol | 2.5% | 2.5% |
| Preservative, fragrance | q.s. | q.s. |
| Citric acid/sodium | q.s. pH 6.5 | q.s. pH 6.5 |
| hydroxide | | |
| Water | q.s. for | q.s. for 100% |
| | 100% | |

Two grams of each composition A and B are applied to locks of sensitive hair with a length of 20 cm and a weight of 2.7 grams. The locks are massaged, left to stand for 5 minutes and then rinsed. The tonicity of the curl, the feel of the hair and the disentangling of the locks using a comb are evaluated. The control hair was not subjected to any treatment.

It is noted that the two compositions according to the invention A and B contribute a virtually identical level of tonicity to the curl; this level is much greater than that obtained with the control lock which is not subjected to any treatment.

### EXAMPLE 4

The constituents of the vessel heel are introduced into a 500 ml reactor equipped with a reflux condenser, a stirrer and a 250 ml dropping funnel. The mixture is heated to 80° C. in 1 hour and then the constituents of the material to be run in are added in 1 hour. The temperature is maintained at 80° C. for 5 hours.

| | Vessel | | Material to | | Total | |
|---|---|---|---|---|---|---|
| | heel | | be run in | | amount | |
| | (g) | | (g) | | (g) | |
| Dimethylaminoethyl | — | | 50 | | 50 | |
| methacrylate | | | | | | |
| Neutralizing agent | — | | 50 | | 50 | |
| (ethylcaproic acid) | | | | | | |
| Stabilizing agent* | 2.5 | | 2.5 | | 5 | |
| Isopropyl myristate | 100 | ml | 100 | ml | 200 | ml |

| | | | |
|---|---|---|---|
| Initiator | — | 1 | 1 |
| (Trigonox 21S) | | | |

*Pecosil FSH-150 from Phoenix Chemical

A dispersion of polymer in isopropyl myristate having a dry matter content of 32% is obtained. The hydrophilic cationic monomer represents 100% by weight of the total weight of monomers.

### EXAMPLE 5

The constituents of the vessel heel are introduced into a 500 ml reactor equipped with a reflux condenser, a stirrer and a 250 ml dropping funnel. The mixture is heated to 80° C. in 1 hour and then the constituents of the material to be run in are added in 1 hour. The temperature is maintained at 80° C. for 5 hours.

| | Vessel | | Material to | | | |
|---|---|---|---|---|---|---|
| | heel | | be run in | | Total | |
| | (g) | | (g) | | amount (g) | |
| Dimethylaminoethyl | 7.5 | | 35 | | 42.5 | |
| methacrylate | | | | | | |
| Neutralizing agent | 7.5 | | 35 | | 42.5 | |
| (ethylcaproic acid) | | | | | | |
| 2-Ethylhexyl acrylate | 7.5 | | 7.5 | | 15 | |
| Stabilizing agent* | 2.5 | | 2.5 | | 5 | |
| Isopropyl myristate | 100 | ml | 100 | ml | 200 | ml |

| | | | |
|---|---|---|---|
| Initiator (Trigonox 21S) | — | 1 | 1 |

*Pecosil FSH-150 from Phoenix Chemical

A dispersion of polymer in isopropyl myristate having a dry matter content of 33% is obtained. The hydrophilic cationic monomer represents 74% by weight of the total weight of monomers.

### EXAMPLE 6

The following compositions (in %) are prepared:

| | Composition | | Composition E |
|---|---|---|---|
| | C | Composition D | Comparative |
| Dispersion of Example 4 | 1% AM | — | — |
| | of polymer | | |
| Dispersion of Example 5 | — | 1% AM of | — |
| | | polymer | |
| Cetyl alcohol | 3% AM | 3% AM | 3% AM |
| Cetyl esters (mixture | 0.25% AM | 0.25% AM | 0.25% AM |
| of myristyl, cetyl and | | | |
| stearyl myristate, | | | |
| palmitate and | | | |
| stearate) | | | |
| Myristyl alcohol | 0.75% AM | 0.75% AM | 0.75% AM |
| Palm oil | 0.15% AM | 0.15% AM | 0.15% AM |
| Hydroxyethylcellulose | 0.2% AM | 0.2% AM | 0.2% AM |
| (MW = 1 300 000) | | | |
| Cetyltrimethylammonium | — | — | 0.45% AM |
| chloride | | | |
| Stearylamidopropyl- | — | — | 0.75% AM |
| dimethylamine | | | |
| Preservative | q.s. | q.s. | q.s. |
| Water | q.s. for | q.s. for | q.s. for 100% |
| | 100% | 100% | |

Two grams of each composition C, D and E are applied to locks of sensitive hair with a length of 20 cm and a weight of 2.7 grams. The locks are massaged, left to stand for 5 minutes then rinsed. The tonicity of the curl, the feel of the hair and the disentangling of the locks using a comb are evaluated.

It is noted that the two compositions according to the invention C and D contribute a virtually identical level of tonicity to the curl; this level is much greater than that obtained with the reference lock treated with the comparative composition E.

1-25. (canceled)

26. A dispersion of particles of at least one ethylenic polymer, stabilized by a stabilizing agent, in a nonaqueous medium comprising of at least one nonaqueous compound which is liquid at 25° C. and which has an overall solubility parameter, according to the Hansen solubility space, of less than or equal to 20 (MPa)^{1/2}, wherein said at least one ethylenic polymer comprises from 50 to 100% by weight of at least one hydrophilic cationic monomer having a log p of less than or equal to 2, with respect to the total weight of starting monomers.

27. The dispersion according to claim 26, wherein the at least one ethylenic polymer comprises from 51 to 99.5% by weight, of the at least one hydrophilic cationic monomer, with respect to the total weight of starting monomers.

28. The dispersion according to claim 27, wherein the at least one ethylenic polymer comprises from 65 to 85% by weight, of the at least one hydrophilic cationic monomer, with respect to the total weight of starting monomers.

29. The dispersion according to claim 26, wherein the at least one hydrophilic cationic monomer exhibits a log p value ranging from −10 to 2.

30. The dispersion according to claim 29, wherein the at least one hydrophilic cationic monomer exhibits a log p value ranging from −2.5 to 1.5.

31. The dispersion according to claim 26, wherein the at least one hydrophilic cationic monomer is chosen from the monomers of following formulae (I) and (II), and their salts: 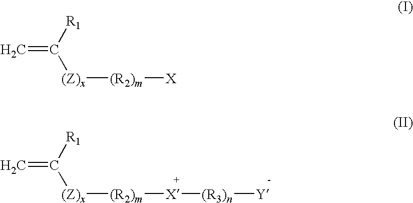
wherein:
R₁ is a hydrogen atom or a linear or branched hydrocarbon radical of CₚH₂ₚ₊₁ type, with p being an integer ranging from 1 to 12;
Z is a divalent group chosen from —COO—, —CONH—, —CONCH₃—, —OCO—, —O—, —SO₂—, —CO—O—CO— or —CO—CH₂—CO—;
x is 0 or 1;
R₂ and R₃ are, independently of one another, a saturated or unsaturated, linear, branched or cyclic, divalent carbon-comprising radical of 1 to 30 carbon atoms, which optionally can comprise from 1 to 18 heteroatoms chosen from O, N, S, F, Si and P;
m is 0 or 1;
n is an integer ranging from 1 to 100;
X is:
(a) a guanidino group or an amidino group; or
(b) a group of formula —N(R₆)(R₇) with R₆ and R₇ representing, independently of one another, (i) a hydrogen atom, (ii) a saturated or unsaturated, optionally aromatic, linear, branched or cyclic alkyl group comprising from 1 to 18 carbon atoms which can optionally comprise from 1 to 10 heteroatoms chosen from O, N, S, F, Si and P; or (iii) R₆ and R₇ form, with the nitrogen atom to which they are attached, a saturated or unsaturated ring of formula: 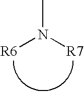
comprising in total from 5 to 8 atoms and optionally from 1 to 4 additional heteroatoms chosen from O, S and N; said ring optionally being fused to at least one another saturated or unsaturated ring comprising 5 to 7 atoms and optionally 2 to 4 heteroatoms chosen from O, S and N;
(c) a ring: 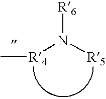
wherein R′₄ and R′₅ form, with the nitrogen atom to which they attached to, a saturated or unsaturated ring comprising in total from 5 to 8 atoms and optionally from 1 to 4 additional heteroatoms chosen from O, S and N; said ring optionally being fused to at least one another saturated or unsaturated ring comprising 5 to 7 atoms and optionally 2 to 4 heteroatoms chosen from O, S and N; and R′₆ is chosen from H, —CH₃ and —C₂H₅;
X′⁺ is a divalent group of formula —N⁺(R₆)(R₇)— with R₆ and R₇ representing, independently of one another, (i) a hydrogen atom, (ii) an optionally aromatic, linear, branched or cyclic alkyl group comprising from 1 to 25 carbon atoms and optionally from 1 to 20 heteroatoms chosen from O, N, S and P; or (iii) R₆ and R₇ form, with the nitrogen atom to which they are attached, a saturated or unsaturated, ring of formula: 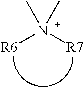
comprising in total from 5 to 8 atoms and optionally from 1 to 4 additional heteroatoms chosen from O, S and N; said ring optionally being fused to at least one another saturated or unsaturated rings comprising from 5 to 7 atoms and optionally from 2 to 4 heteroatoms chosen from O, S and N;
or else X′⁺ is a divalent ring of formula: 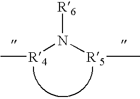
wherein R′₄ and R′₅ form, with the nitrogen atom to which they are attached to, a saturated or unsaturated ring comprising in total from 5 to 8 atoms and optionally from 1 to 4 additional heteroatoms chosen from O, S and N; said ring optionally being fused to at least another saturated or unsaturated ring comprising from 5 to 7 atoms and optionally from 2 to 4 heteroatoms chosen from O, S and N; and R′₆ is chosen from H, —CH₃ and —C₂H₅;
Y′⁻ is a group chosen from —COO⁻, —SO₃⁻, —OSO₃⁻, —PO₃²⁻ and —OPO₃²⁻.

32. The dispersion according to claim 31, wherein Z is chosen from COO and CONH.

33. The dispersion according to claim 31, wherein n is an integer ranging from 1 to 5.

34. The dispersion according to claim 31, wherein X is chosen from:
a group of formula —N(R₆)(R₇) with R₆ and R₇ representing, independently of one another, (i) a hydrogen atom, (ii) a saturated or unsaturated, linear, branched or cyclic alkyl group comprising from 1 to 18 carbon atoms, or (iii) R₆ and R₇ form, with the nitrogen atom to which they are attached, a saturated or unsaturated ring of formula: 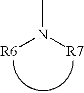
comprising in total from 5 to 7 atoms and optionally from 1 to 3 additional heteroatoms chosen from O, S and N;
a ring: 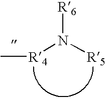
wherein R′₄ and R′₅ form, with the nitrogen atom to which they are attached to, a saturated or unsaturated ring comprising from 5 to 7 atoms.

35. The dispersion according to claim 31, wherein X is chosen from an NH₂, N(CH₃)₂, pyridinyl, imidazolyl, piperidinyl, piperazinyl or morpholinyl group.

36. The dispersion according to claim 26, wherein the at least one hydrophilic cationic monomer is chosen from:
dimethylaminopropyl(meth)acrylamide, dimethylaminoethyl(meth)acrylamide, diethylaminopropyl(meth)acrylamide;
diethylaminoethyl (meth)acrylate, dimethylaminoethyl (meth)acrylate, morpholinoethyl (meth)acrylate or tert-butylaminoethyl (meth)acrylate;
vinylimidazole, vinylpyridine, vinylamine,
and the following monomers: 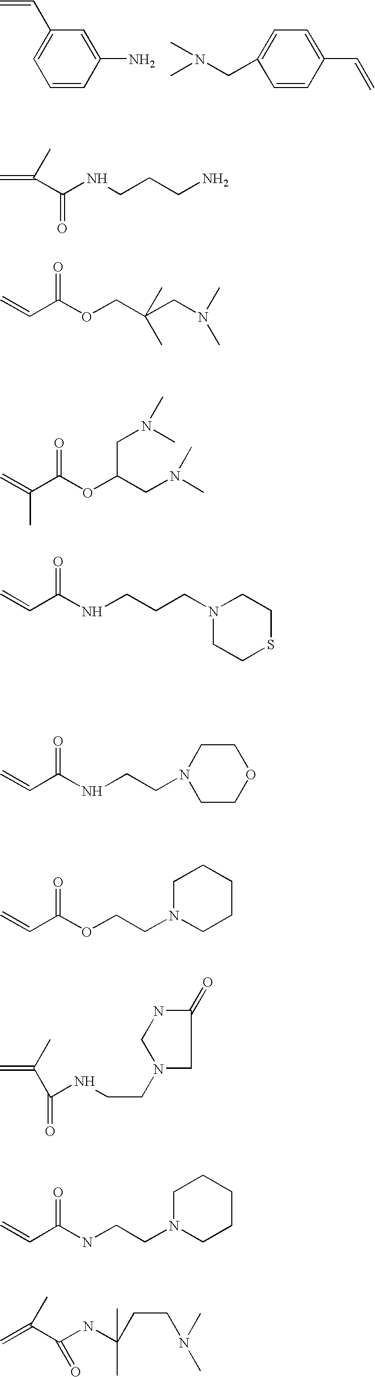 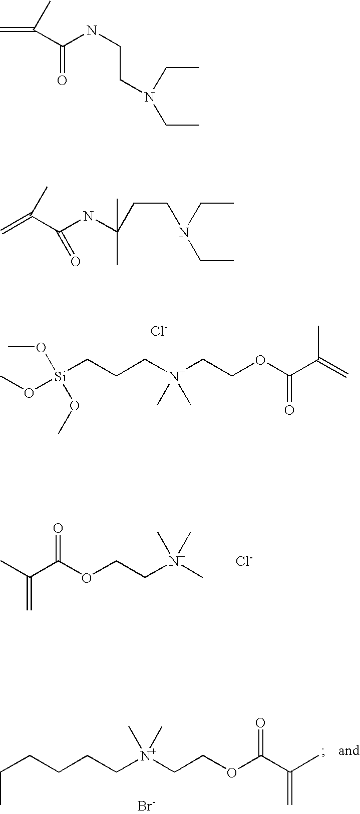
N,N-dimethyl-N-(2-methacryloyloxyethyl)-N-(3-sulphopropyl)ammonium betaine, N,N-dimethyl-N-(3-methacrylamidopropyl)-N-(3-sulphopropyl)ammonium betaine, 1-(3-sulphopropyl)-2-vinylpyridinium betaine and 2-methacryloyloxyethyl phosphorylcholine.

37. The dispersion according to claim 26, wherein the at least one hydrophilic cationic monomer is chosen from:
dimethylaminopropyl(meth)acrylamide or dimethylaminoethyl(meth)acrylamide,
diethylaminoethyl (meth)acrylate, dimethylaminoethyl (meth)acrylate, morpholinoethyl (meth)acrylate,
vinylimidazole or vinylpyridine, and
the following monomers:

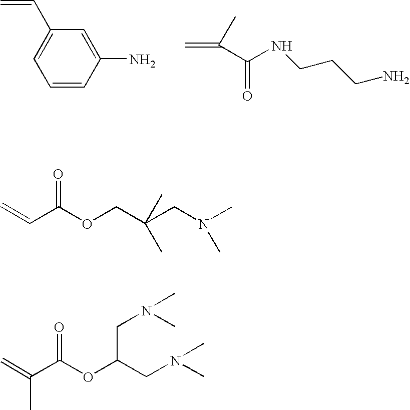

38. The dispersion according to claim 26, wherein the at least one ethylenic polymer comprises 100% by weight of at least one hydrophilic cationic monomer, with respect to the total weight of the starting monomers.

39. The dispersion according to claim 26, wherein the at least one ethylenic polymer further comprises at least one additional monomer present in an amount of 0.01 to 50% by weight, with respect to the total weight of starting monomers.

40. The dispersion according to claim 39, wherein the at least one ethylenic polymer further comprises at least one additional monomer present in an amount of 15 to 35% by weight, with respect to the total weight of starting monomers.

41. The dispersion according to claim 39, wherein the at least one additional monomer is chosen from:
(i) (meth)acrylic acid esters of formula CH₂═CHCOOR₄ or CH₂═C(CH₃)COOR₄ with R₄ representing a saturated or unsaturated, linear, cyclic or branched carbon-comprising chain, having from 1 to 32 carbon atoms and optionally comprising at least one heteroatom chosen from O, N, S and/or optionally substituted by at least one substituent chosen from —OH and halogen atoms;
(ii) (meth)acrylamides of formula CH₂═CHCONR₅R′₅ or CH₂═C(CH₃)CONR₅R′₅ wherein R₅ and R′₅, which are identical or different, represent a hydrogen atom or a saturated or unsaturated, linear, cyclic or branched carbon-comprising chain, having from 6 to 28 carbon atoms and optionally comprising at least one heteroatom chosen from O, N and S and/or optionally substituted by at least one substituent chosen from —OH and halogen atoms;
(iii) vinyl esters of formula CH₂═CH—OCO—R₆ with R₆ representing a saturated or unsaturated, linear or branched, carbon-comprising chain, having from 1 to 12 carbon atoms;
(iv) vinyl ethers of formula CH₂═CHOR₇ with R₇ representing a saturated or unsaturated, linear or branched carbon-comprising chain, having from 1 to 12 carbon atoms;
(v) vinyl compounds of formula CH₂═CHR₈ wherein R₈ is
a hydroxyl group;
a linear or branched alkyl group comprising from 1 to 25 carbon atoms wherein at least one heteroatom chosen from O, N, S and P is optionally inserted; said alkyl group optionally being substituted by at least one substituent chosen from —OH and halogen atoms;
a C₃ to C₈ cycloalkyl group,
a C₆ to C₂₀ aryl group,
a C₇ to C₃₀ aralkyl group wherein the alkyl is C₁-C₄ alkyl,
a 4- to 12-membered heterocyclic group comprising at least one heteroatom chosen from O, N and S,
a heterocycloalkyl group wherein the alkyl is C₁-C₄ alkyl,
wherein said cycloalkyl, aryl, aralkyl, heterocyclic or heterocycloalkyl groups optionally are substituted by at least one substituent chosen from the hydroxyl group, halogen atoms and linear and branched C₁-C₄ alkyl groups wherein at least one heteroatom chosen from O, N, S and P is optionally inserted, said alkyl groups being optionally substituted by at least one substituents chosen from —OH and halogen atoms;
(vi) multifunctional monomers;
(vii) (meth)acrylic acid, maleic anhydride, crotonic acid, itaconic acid, fumaric acid, maleic acid, diacrylic acid, dimethylfumaric acid, citraconic acid, acrylamidopropanesulphonic acid, 2-acrylamido-2-methylpropanesulphonic acid, styrenesulphonic acid, vinylbenzoic acid, vinylphosphoric acid, vinylsulphonic acid, vinylbenzenesulphonic acid, acrylamidoglycolic acid of formula CH₂═CH—CONHCH(OH)COOH, vinylphosphonic acid, sulphopropyl (meth)acrylate, sulphoethyl (meth)acrylate, vinyl methyl sulphone, 2-(methacryloyloxy)ethyl phosphate of formula CH₂═C(CH₃)COOC₂H₄OP(O)(OH)₂, and salts thereof; and
(viii) poly(ethylene glycol) (meth)acrylates, alkyl poly(ethylene glycol) (meth)acrylates.

42. The dispersion according to claim 40, wherein the at least one additional monomer is chosen from (meth)acrylic acid C₁-C₁₈ esters, (meth)acrylamides, styrene, ethylhexyl vinyl ether, dodecyl vinyl ether, vinyl hexanoate, and 2-hydroxyethyl acrylate and poly(ethylene glycol) methacrylate.

43. The dispersion according to claim 42, wherein the (meth)acrylic acid C₁-C₁₈ esters are chosen from methyl, ethyl, propyl, n-butyl, isobutyl, 2-ethylhexyl, tert-butyl, isooctyl, decyl, myristyl and stearyl (meth)acrylates.

44. The dispersion according to claim 42, wherein the (meth)acrylamides are chosen from methyl-, ethyl-, propyl-, (n-butyl)-, isobutyl-, (2-ethylhexyl)-, (tert-butyl)-, isooctyl-, decyl-, myristyl- and stearyl(meth)acrylamides.

45. The dispersion according to claim 26, wherein the at least one nonaqueous liquid compound having an overall solubility parameter according to the Hansen solubility space of less than or equal to 20 (MPa)^{1/2} is chosen from liquid fatty substances.

46. The dispersion according to claim 45, wherein the liquid fatty substances comprise optionally branched, carbon-comprising, hydrocarbon, fluorinated and/or silicone, natural or synthetic oils.

47. The dispersion according to claim 26, wherein the at least one nonaqueous liquid compound is chosen from:
vegetable oils comprising esters of fatty acids and of polyolsor esters derived from long-chain C₆-C₂₀ acids or alcohols;
hydrocarbons;
silicone oils optionally substituted by optionally fluorinated aliphatic and/or aromatic groups or by functional groups and volatile silicone oils;
solvents chosen from linear, branched and cyclic esters having from 6 to 30 carbon atoms, ethers having from 6 to 30 carbon atoms and ketones having from 6 to 30 carbon atoms; and
fatty aliphatic monoalcohols having at least 6 carbon atoms, the hydrocarbon chain not comprising a substituent group.

48. The dispersion according to claim 47, wherein the vegetable oils comprise triglycerides.

49. The dispersion according to claim 47, wherein the vegetable oils comprise sunflower, sesame or rapeseed oil.

50. The dispersion according to claim 47, wherein the vegetable oils comprises esters of formula RCOOR′ wherein R represents the residue of a higher fatty acid comprising from 7 to 19 carbon atoms and R′ represents a hydrocarbon chain comprising from 3 to 20 carbon atoms.

51. The dispersion according to claim 26, wherein the at least one nonaqueous liquid compound is chosen from volatile silicone oils and esters of formula RCOOR′ wherein R represents the residue of a higher fatty acid comprising from 7 to 19 carbon atoms and R′ represents a hydrocarbon chain comprising from 3 to 20 carbon atoms.

52. The dispersion according to claim 51, wherein the volatile silicone oils comprise cyclodimethylsiloxanes or linear dimethylsiloxanes.

53. The dispersion according to claim 51, wherein the at least one nonaqueous liquid compound is chosen from palmitates, adipates, myristates and benzoates, diisopropyl adipate and isopropyl myristate.

54. The dispersion according to claim 26, wherein the stabilizing agent is chosen from sequential polymers, grafted polymers and random polymers, alone or as a blend.

55. The dispersion according to claim 26, wherein the stabilizing agent is present in an amount ranging from 0.1 to 30% by weight, with respect to the weight of the starting monomers.

56. The dispersion according to claim 55, wherein the stabilizing agent is present in an amount ranging from 3 to 10% by weight, with respect to the weight of the starting monomers.

57. The dispersion according to claim 26, wherein the stabilizing agent is chosen from:
silicone polymers grafted with a hydrocarbon chain and hydrocarbon polymers grafted with a silicone chain;
grafted copolymers having an insoluble backbone of polyacrylic type with soluble grafts of poly(12-hydroxystearic acid) type;
grafted or sequential block copolymers comprising at least one block of polyorganosiloxane type and at least one block of a polymer (i) resulting from radical polymerization or (ii) resulting from polycondensation;
grafted or sequential block copolymers of C₁-C₄ alkyl (meth)acrylates and of C₈-C₃₀ alkyl (meth)acrylates;
grafted or sequential block copolymers comprising at least one block resulting from the polymerization of ethylenic monomer, comprising at least one optionally conjugated ethylenic bond, and/or dienes; and at least one block of polymer resulting from radical polymerization other than of dienes;
alkyl dimethicones wherein the alkyl group comprises 6 to 32 carbon atoms;
dimethiconol esters of formula: 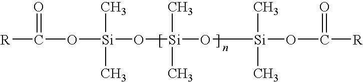
wherein R is an alkyl radical having from 6 to 32 carbon atoms;
alkylamidoamines having from 6 to 60 carbon atoms;
copolymers comprising at least one polyorganosiloxane part and fluorinated groups, of formula: 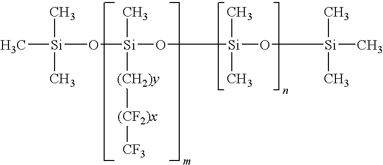
wherein x is an integer ranging from 3 to 12; y is an integer ranging from 2 to 6; and m and n are such that the molecular weight of the compound ranges from 5000 to 15,000.

58. The dispersion according to claim 57, wherein the grafted or sequential block copolymers comprising at least one block of polyorganosiloxane type and at least one block of a polymer (i) resulting from radical polymerization or (ii) resulting from polycondensation of polyether, polyester or polyamide type, or their blend, wherein said copolymer is optionally fluorinated.

59. The dispersion according to claim 26, wherein the stabilizing agent is chosen from:
fluorinated silicones or fluorosilicones of formula: 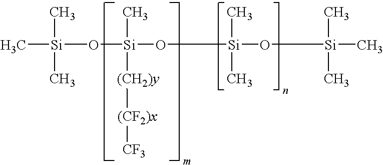
wherein x=8, y=2 or 3, and m and n are such that the molecular weight of the compound ranges from 5000 to 15,000; and
grafted and sequential block copolymers comprising at least one block of polyorganosiloxane type and at least one block of a polymer resulting from polycondensation.

60. The dispersion of claim 59, wherein the grafted and sequential block copolymers comprising at least one block of polyorganosiloxane type and at least one block of a polymer resulting from polycondensation of polyether, polyester or polyamide type.

61. The dispersion of claim 60, wherein the grafted and sequential block copolymers comprising at least one block of polyorganosiloxane type and at least one block of a polymer resulting from polycondensation of polyoxyethylene and/or polyoxypropylene type.

62. A cosmetic or pharmaceutical composition comprising, in a cosmetically or pharmaceutically acceptable medium, at least one dispersion of particles of at least one ethylenic polymer, stabilized by a stabilizing agent, in a nonaqueous medium comprising at least one nonaqueous compound which is liquid at 25° C. and which has an overall solubility parameter, according to the Hansen solubility space, of less than or equal to 20 (MPa)^{1/2}, wherein said at least one ethylenic polymer comprises from 50 to 100% by weight of at least one hydrophilic cationic monomer having a log p of less than or equal to 2, with respect to the total weight of starting monomers.

63. The cosmetic or pharmaceutical composition according to claim 62, further comprising at least one constituent chosen from waxes, oils, gums, and pasty fatty substances of vegetable, animal, mineral and synthetic, and silicone origin; colouring materials chosen from pulverulent compounds, fat-soluble, and water-soluble dyes; antioxidants; fragrances; essential oils; preservatives; cosmetic active principles; moisturizing agents; vitamins; essential fatty acids; ceramides; sunscreens; surfactants; polymers; thickeners, and gelling agents.

64. The cosmetic or pharmaceutical composition according to claim 62, provided in the form of a suspension, a dispersion, by virtue of vesicles; an optionally gelled, oily solution; an oil-in-water, water-in-oil or multiple emulsion; a gel or a foam; an oily or emulsified gel; a dispersion of vesicles; a two-phase or multiphase lotion; a spray; a lotion; a cream; an ointment; a soft paste; a salve; a cast or moulded solid; or a compacted solid.

65. The cosmetic or pharmaceutical composition according to claim 64, provided in the form of a dispersion of oil in water.

66. The cosmetic or pharmaceutical composition according to claim 64, provided in the form of a dispersion of lipid vesicles.

67. The cosmetic or pharmaceutical composition according to claim 64, provided in the form of a cast or moulded solid as a stick or in a dish.

68. The cosmetic or pharmaceutical composition according to claim 62, provided in the form of a product for caring for and/or making up the skin of the body or face, the lips and the hair; of an antisun or self-tanning product; or of a hair product for the treatment, washing, form retention or shaping of the hair.

69. The cosmetic or pharmaceutical composition according to claim 62, provided in the form of a shampoo, gel, hairsetting lotion, blow-drying lotion, or fixing and styling composition; of a shower gel or of a foam bath; of a rinse-out or leave-in conditioner, of a perming, hair-straightening, dyeing or bleaching composition; of a rinse-out composition, to be applied before or after dyeing, bleaching, perming or hair straightening or else between the two stages of a perming or hair straightening; of washing compositions for the skin; of aqueous or aqueous/alcoholic lotions for caring for the skin and/or hair; or of a hair care product.

70. A method for the cosmetic treatment of keratinous substances, comprising the application, to said keratinous substances, of a cosmetic composition, comprising, in a cosmetically or pharmaceutically acceptable medium, at least one dispersion of particles of at least one ethylenic polymer, stabilized by a stabilizing agent, in a nonaqueous medium comprising at least one nonaqueous compound which is liquid at 25° C. and which has an overall solubility parameter, according to the Hansen solubility space, of less than or equal to 20 (MPa)^{1/2}, wherein said at least one ethylenic polymer comprises from 50 to 100% by weight of at least one hydrophilic cationic monomer having a log p of less than or equal to 2, with respect to the total weight of starting monomers.
